(19) United States
(12) Patent Application Publication
(10) Pub. No.: US 2006/0234382 A1
(43) Pub. Date: Oct. 19, 2006

(54) System and Method for Predicting Mold Growth in an Environment

(75) Inventors: Jianrong Wang, Sugar Land, TX (US); Chaoming Zhang, Sugar Land, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: IAQ Laboratories International, LLC, Tavernier, FL (US)

(21) Appl. No.: 11/379,250

(22) Filed: Apr. 19, 2006

Related U.S. Application Data
(60) Provisional application No. 60/672,812, filed on Apr. 19, 2005.

(51) Int. Cl. G01N 33/00

(52) U.S. Cl. 436/39

(57) ABSTRACT

Mold growth monitoring and prediction systems and methods for an environment are disclosed. The system includes a processing unit, a temperature sensor, and a humidity sensor. The processing unit obtains a temperature reading and a humidity reading of the environment from the sensors. The processing unit uses an algorithm to determine a probability of mold growth based on the temperature reading, the humidity reading, and a time reading. For example, the algorithm defines an envelope based on temperature, humidity, and one or more species of mold. The envelope substantially separates conditions detrimental to mold growth from conditions conducive to mold growth for the species of mold. The processing unit uses the algorithm to determine whether the temperature reading and the humidity reading fall within detrimental or conducive conditions to mold growth. Based on the conditions, the processing unit either increases or decreases the probability of mold growth.

### CROSS-REFERENCE TO RELATED APPLICATIONS

This is a non-provisional of U.S. Provisional Application Ser. No. 60/672,812, filed Apr. 19, 2005, which is incorporated herein by reference and to which priority is claimed.

### FIELD OF THE DISCLOSURE

The subject matter of the present disclosure generally relates to a system and method for predicting mold growth in an environment and more particularly relates to a system and method for monitoring temperature and humidity conditions of an environment and determining a probability of mold growth in the environment to produce a mold warning and/or to operate an environmental system to decrease the potential mold growth.

### BACKGROUND OF THE DISCLOSURE

Molds are members of the kingdom fungi and live extensively throughout nature. Molds can grow indoors and can cause various health risks or environmental damage. Molds have three phases of growth, which include spore germination, mycelium growth, and sporulation. Four conditions (temperature, humidity, nutrients, and time) contribute to the potential for mold growth in an environment. Typical indoor environments where mold grows include moist basements, bathrooms, kitchens, or any place where moisture is present. Mold only requires a few nutrients and can grow on various substrates, including, but not limited to, wood, ceiling tiles, gypsum wallboard (sheetrock), cardboard, paper, cellulosic surfaces, carpet, etc.

The influence of temperature, relative humidity, nutrients, and time on mold growth is known in the art. Referring to graphs 10 and 20 of FIG. 1, for example, isopleths 12 and 22 of spore germination for various molds are shown as functions of temperature and relative humidity. The isopleths 12 and 22 are determined from experimental measurements of spore germination for species of mold on a given substrate. The isopleths are arranged according to time (e.g., days of 1d, 2d, 4d, 8d, 16d, and LIM) in which a particular level of spore germination occurs (e.g., the length of time after which the first germination occurs at a given temperature and relative humidity). The lowest isopleths (LIM) represent limits of the conditions conducive to spore germination for the given substrate. Below these limits, spore germination does not occur for the mold at the temperature and relative humidity levels.

One technique known in the art to detect mold involves sampling the air in an environment to identify the various types and quantities of mold spores interspersed in the air. A collection device obtains a predetermined amount of air from the environment, and the sample is then analyzed in a laboratory. Another technique known in the art to detect mold involves taking direct samples (e.g., swab or tape-lifted samples) of suspect surfaces to confirm and identify the presence of mold. Direct sampling identifies the types of mold found, but not a spore count. Again, the sample is then analyzed in a laboratory. To detect hidden mold, it is known in the art for an inspector to use a hygrometer, a boroscope (fiber optics), and a moisture meter to find hidden mold behind walls, ceilings and floors, for example, and to determine areas of potential mold growth and continuing moisture penetration.

Unfortunately, the prior art techniques are only effective at detecting mold after it is allowed to develop. Furthermore, there are thousands of species of molds, and the prior art techniques are typically designed to detect only specific species of mold. Therefore, a need exists in the art for a system and method to determine proactively the probability of growth of one or more species of mold in an environment and to control proactively the conditions of the environment to reduce or reverse mold growth.

The subject matter of the present disclosure is directed to overcoming, or at least reducing the effects of, one or more of the problems set forth above.

### SUMMARY OF THE DISCLOSURE

Mold growth prediction systems and methods for an environment are disclosed. The system includes a processing unit, a temperature sensor, and a humidity sensor. The processing unit has an interface for obtaining a temperature reading and a humidity reading of the environment from the sensors. The processing unit also has a memory for storing an algorithm to determine a probability of mold growth and has a processor communicatively coupled to the interface and the memory. The processor processes the temperature reading, the humidity reading, and a time reading according to the algorithm to determine the probability of mold growth. For example, the algorithm defines an envelope based on temperature, humidity, and one or more species of mold. The envelope substantially separates conditions detrimental to mold growth from conditions conducive to mold growth for the species of mold. The processor uses the algorithm to determine whether the temperature reading and the humidity reading fall within detrimental or conducive conditions to mold growth. Based on the conditions, the processor either increases or decreases the probability of mold growth, and the processor can then controls an environmental system to address the mold growth.

The foregoing summary is not intended to summarize each potential embodiment or every aspect of the present disclosure.

### BRIEF DESCRIPTION OF THE DRAWINGS

The foregoing summary, preferred embodiments, and other aspects of subject matter of the present disclosure will be best understood with reference to a detailed description of specific embodiments, which follows, when read in conjunction with the accompanying drawings, in which:

FIG. 1 illustrates generalized isopleths of spore germination and mycelium growth for one kind of mold.

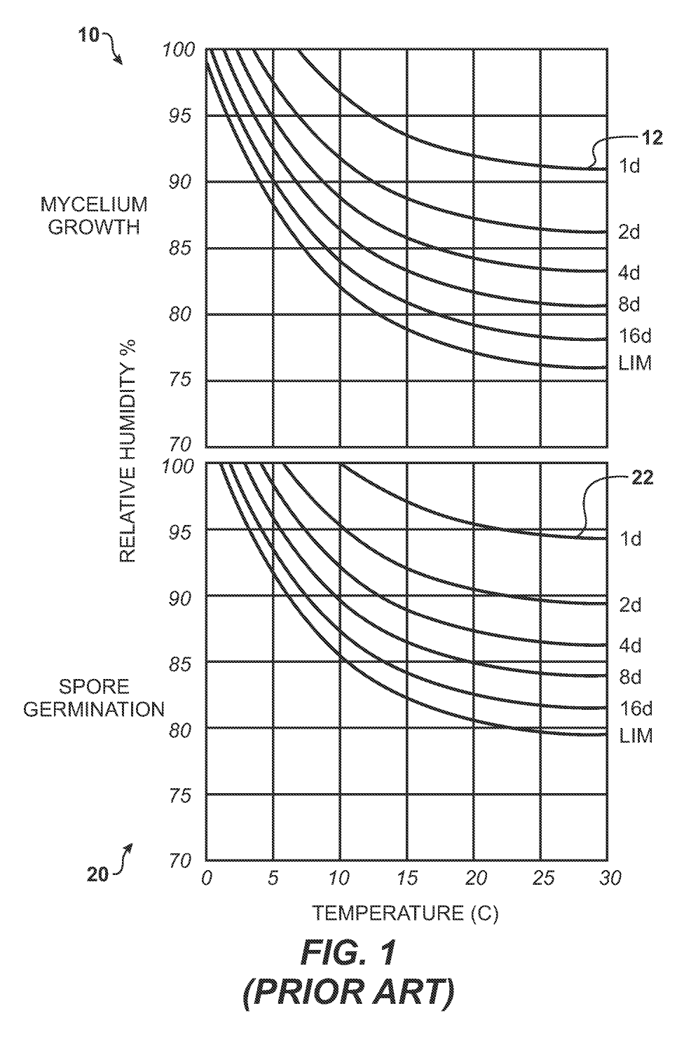

FIG. 2 illustrate a schematic view of conditions (relative humidity, temperature, quality, and time), which can be used to determine the probability of growth for mold.

FIG. 3 illustrates an embodiment of a mold growth prediction system according to certain teachings of the present disclosure.

FIG. 4 illustrates the monitoring unit for the mold growth prediction system of FIG. 3.

FIG. 5 illustrates another embodiment of a mold growth prediction system for an environment according to certain teachings of the present disclosure.

FIG. 6 illustrates graphs showing isopleths for different species of mold.

FIG. 7 illustrates an embodiment of the operation of the disclosed prediction system.

FIGS. 8A-8B graphically illustrate examples of sensor readings and calculated values for mold growth risk factor.

FIGS. 9A-9B graphically illustrate additional examples of sensor readings and calculated values for mold growth risk factor.

FIGS. 10A-10B illustrate example screens of a user interface for a master control computer.

FIG. 11 illustrates an embodiment of an integrated monitoring and environmental system according to certain teachings of the present disclosure.

FIG. 12 illustrates an embodiment of the operation of integrated monitoring and environmental system of FIG. 11.

While the disclosed systems and methods are susceptible to various modifications and alternative forms, specific embodiments thereof have been shown by way of example in the drawings and are herein described in detail. The figures and written description are not intended to limit the scope of the inventive concepts in any manner. Rather, the figures and written description are provided to illustrate the inventive concepts to a person skilled in the art by reference to particular embodiments, as required by 35 U.S.C. § 112.

### DETAILED DESCRIPTION

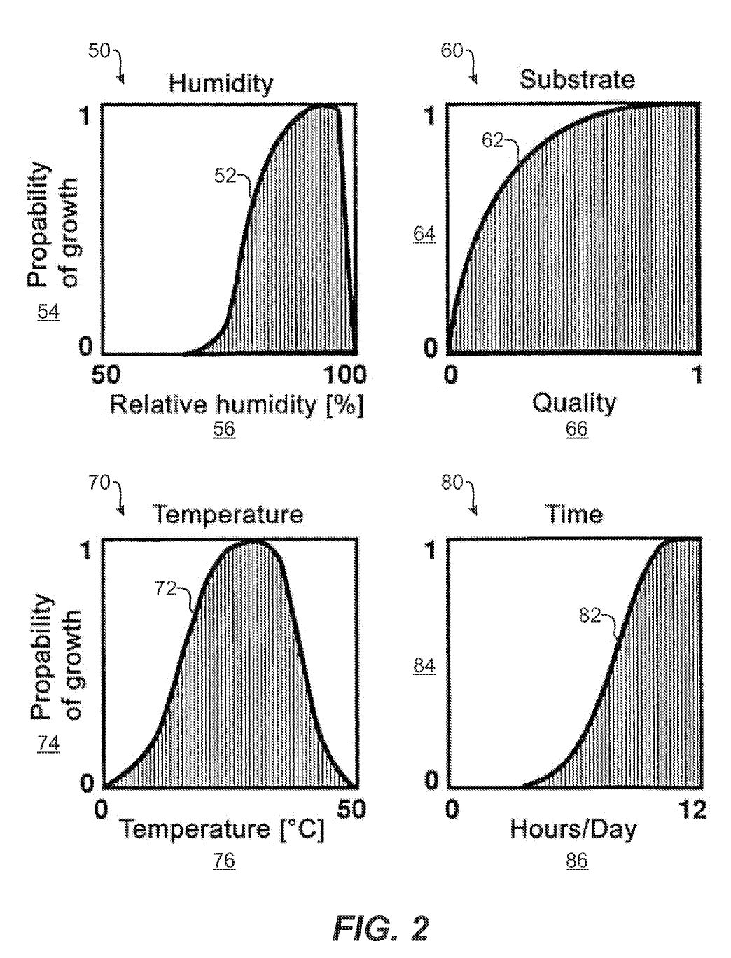

Referring to FIG. 2, graphs schematically show how four conditions (i.e., relative humidity, temperature, quality, and time) that influence mold growth can be used to determine the probability of mold growth. In the humidity graph 50, for example, curve 52 shows how the probability 54 of mold growth corresponds to relative humidity 56. As shown by curve 52, the probability 54 is practically non-existent when the relative humidity 56 is close to fifty-percent, but the probability 54 increases as the relative humidity 56 is closer to one-hundred percent. At a level of relative humidity 56 quite close to one-hundred percent, the probability 52 of mold growth decreases sharply.

In the substrate graph 60, for example, curve 62 shows how the probability 64 of mold growth corresponds to the quality 66 of the substrate on which the mold grows. The quality 66 of the substrate refers to the quality of the material that the mold can use for nutrients. Typical substrates include carpet, wood, wallpaper, etc. As shown by curve 62, the probability 64 for growth increases with the quality 66 of the substrate.

In the temperature graph 70, for example, curve 72 shows how the probability 74 of mold growth corresponds to the temperature 76 of the environment. As shown by curve 72, the probability 74 for mold growth exhibits a bell-shape, where the highest probability 74 occurs somewhere between 0 and 50-degrees Celsius and the probability 74 tapers towards both upper and lower temperatures.

In the time graph 80, for example, curve 82 shows how the probability 84 of mold growth increases with the passage of time 86 (e.g., hours or days). It will be appreciated that the various graphs 50, 60, 70, and 80 are interdependent such that one condition (e.g., relative humidity) could alter the probability curve of another condition (e.g., time). For example, a high probability due to a conducive level of relative humidity will result in an accelerated time curve for mold growth.

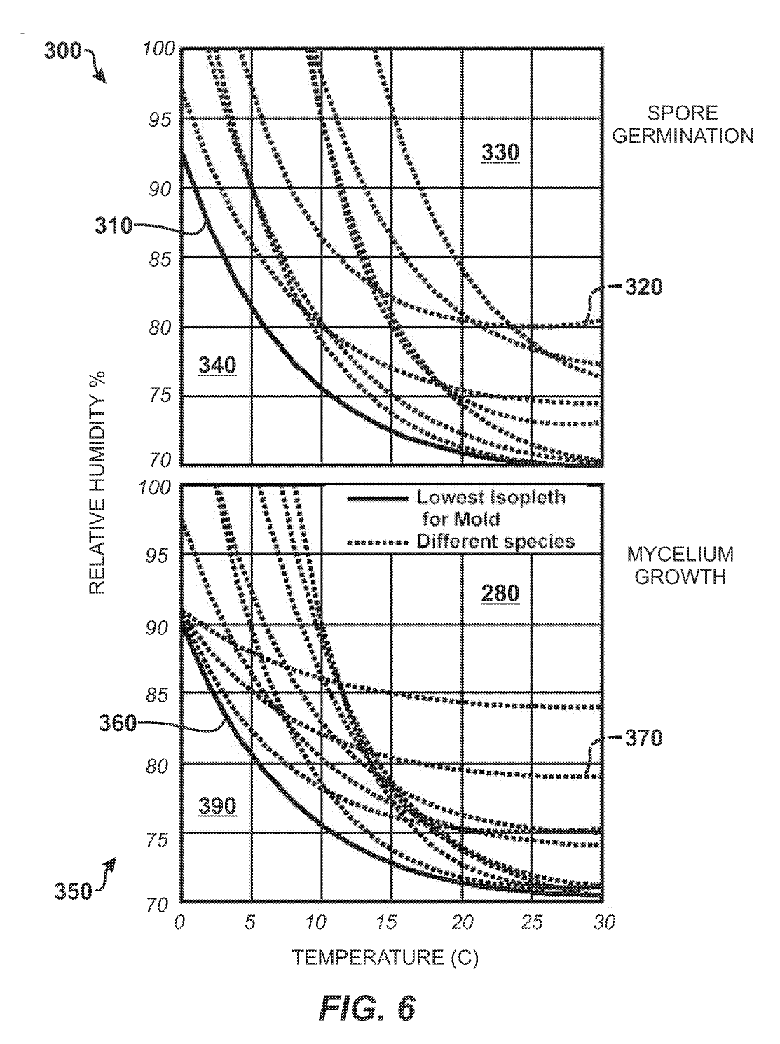

To determine the probability of mold growth in an environment, the systems and methods of the present disclosure incorporate experimental data similar to that shown in FIG. 2. The experimental data captures the interdependence of relative humidity, temperatures, substrates, and time for various species of mold. The types of substrates may be particular for a given environment, or a general nutrient level of substrate may be assumed based on the circumstances. Isopleths, such as discussed below with reference to FIG. 6, for the various species are produced from the experimental data. The information in these isopleths is then analyzed by numerical techniques and then incorporated into an algorithm that can be implemented electronically by a mold prediction system.

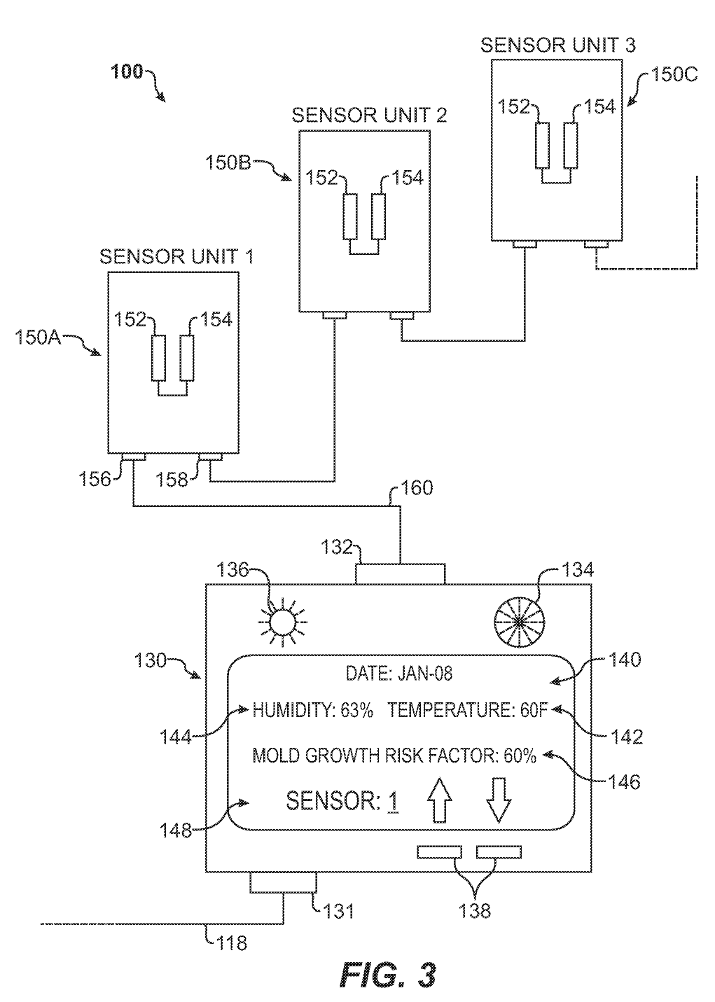

Referring to FIG. 3, an embodiment of a mold growth prediction system 100 according to certain teachings of the present disclosure is illustrated. The prediction system 100 includes one or more monitoring units 130—only one of which is shown in FIG. 3. The prediction system 100 also includes a plurality of sensor units 150 that are distributed throughout an environment. The monitoring unit 130 is communicatively coupled to the sensor units 150, and the monitoring unit 130 uses the sensor units 150 to monitor for conditions (e.g., temperature and relative humidity) conducive to the growth of mold in the environment over intervals of time. (In one embodiment, the temperature and relative humidity readings are taken approximately every 30-minutes, which is believed to generate a sufficient amount of historical data without too much power consumption.) As discussed herein, there is an envelope of conditions conducive to mold growth. If the monitored conditions of a zone or area near sensor units 150 in the environment are within the envelope, then the risks for mold growth are increased for that particular zone. If, however, the monitored conditions of the zone or area are outside the envelope, then the risks for mold growth are reduced for that particular zone or area.

In the present embodiment, the monitoring unit 130 includes an interface 132, a speaker 134, a warning indicator 136, control keys 138, and a display panel 140. The interface 132 is preferably based on the Meter-Bus (“M-Bus”) protocol, which is a European standard used for remotely reading heat-meters and various sensors. The M-Bus offers a number of advantages, including a reduced wiring requirement, individually addressable sensors, and short reading intervals. The interface 132 is communicatively coupled to an input connection terminal 156 of a first of the sensor units 150. This input connection terminal 156 can include connections for ground, VCC, and data connections. An output connection terminal 158 of the first of the sensor units 150 is then connected to another of the sensor units 150. The output connection terminal 158 includes connections for ground, VCC, and data connections. The additional sensor units 150 of the system 100 are then connected serially in this same manner.

The sensor units 150 can be mounted into or onto walls or other structural components of an environment. Each of the sensor units 150 can house both a temperature sensor or thermistor 152 and a relative humidity sensor or hydrometer 154. These sensors 152 and 154 respectively monitor transient states of the temperature and relative humidity conditions near the unit 150 and relay their readings to the monitoring unit 130 via the interface 132. Typically, the temperature and humidity sensors 152 and 154 of the sensor units 150 are sensitive to the resistance and capacitance of the connection circuit. This sensitivity can makes it difficult for the sensor units 150 to be fully exchangeable. Preferably, the sensor units 150 selected for the disclosed system 100 are exchangeable so that the connection may not impact the measurement accuracy.

In a preferred embodiment, the sensors 152 and 154 of the sensor unit 150 are MEMS based sensors from Sensirion, Hygrometrix, and Kelian electronics. For example, suitable Sensirion sensors include Model SHT11 and Model SHT10, which are a single chip relative humidity and temperature multi-sensor module. Suitable Kelian thermistor sensors include Model CL-M52R and Model KL-103-88377. A suitable Hygrometrix sensor includes Model HMX2000-HT.

The monitoring unit 130 may be more or less sophisticated than shown in FIG. 3 depending on the particular implementation of the prediction system 100. In one embodiment of the prediction system 100, for example, the monitoring unit 130 can be a stand-alone device added to a facility or building and capable of independently determining the risk factor for mold growth associated with its connected sensor units 150. The various sensor units 150 can be positioned in rooms or areas where it is desirable to monitor for potential mold growth. The monitoring unit 130 can be positioned in a location where a user can access the unit 130, see the warning indicator 136, hear the speaker 134, and/or use the display panel 140 and control keys 138. In such a stand-alone embodiment, the monitoring unit 130 can collect the sensor readings from the sensor units 150 and can calculate a probability for mold growth or risk factor using an algorithm as disclosed herein. The monitoring unit 130 can then display the calculated mold growth risk factor 146 for a selected zone or sensor unit 150 on the display panel 140.

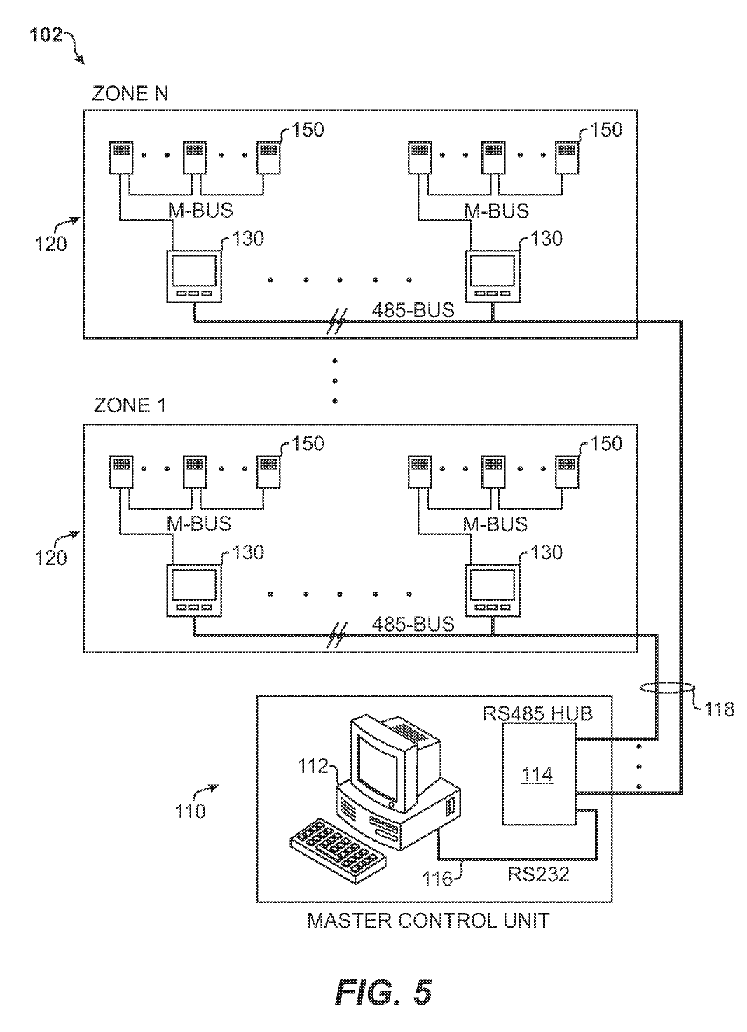

Alternative embodiments and implementations of the disclosed prediction system 100 may not use or require such a stand-alone monitoring unit 130. For example, the various hardware and software components disclosed herein in connection with the monitoring unit 130 can be implemented as or integrated into a computer system, an environmental control system, or a security system. In one alternative embodiment, for example, the monitoring unit 130 can calculate the mold growth risk factor for the areas associated with its connected sensor units 150. Then, the monitoring unit 130 can display the calculated risk factor and/or can send the calculated risk factor to a master control computer via an RS-485 interface 131 and RS-485 Bus 118. (An example of such a master control computer is disclosed below as element 112 of FIG. 5). In yet another alternative embodiment, the monitoring unit 130 can communicate its sensor readings to a master control computer (112; FIG. 5) via the RS-485 interface 131 and RS-485 Bus 118 without first calculating the mold growth risk factor. Then, the master control computer (112; FIG. 5) can determine the mold growth risk factor and can present relevant information, alarms, trends, history, etc. for the user.

Various designs for the display panel 140 on the monitoring unit 130 can be used to display information for users. Among other information (e.g., the date and zone name), the display panel 140 in the present embodiment displays the current temperature condition 142, the current humidity condition 144, and the calculated mold growth risk factor 146 associated with a selected sensor unit identifier 148. The display panel 140 can also show trends, such as temperature trends, humidity trends, and risk factor trends. In one embodiment, the display panel 140 can be a touch screen. Alternatively, the monitoring unit 130 has control keys 138. Using the control keys 138, a user can change the information displayed on the panel 140 or can alter information used by the monitoring unit 130. The speaker 134 can produce a warning sound if the mold growth risk factor 146 for a zone or sensor unit exceeds a predetermined threshold. Similarly, the warning indicator 136 can produce a warning light if such a case occurs.

Additional forms of information can be displayed on the display 140 of the monitoring unit 130. Some examples of information include the number of sensor units 150 connected to the monitoring unit 130, the number of collected sensor records stored in the monitoring unit 130, and the identification number of the monitoring unit 130. The display 140 can also show which sensors have failed to collect data. In addition, various functions may be accessible using the display 140 of the monitoring unit 130. Some example functions include running tests of selected sensor units 150 and setting ID numbers for the monitoring unit 130 and connected sensor units 150.

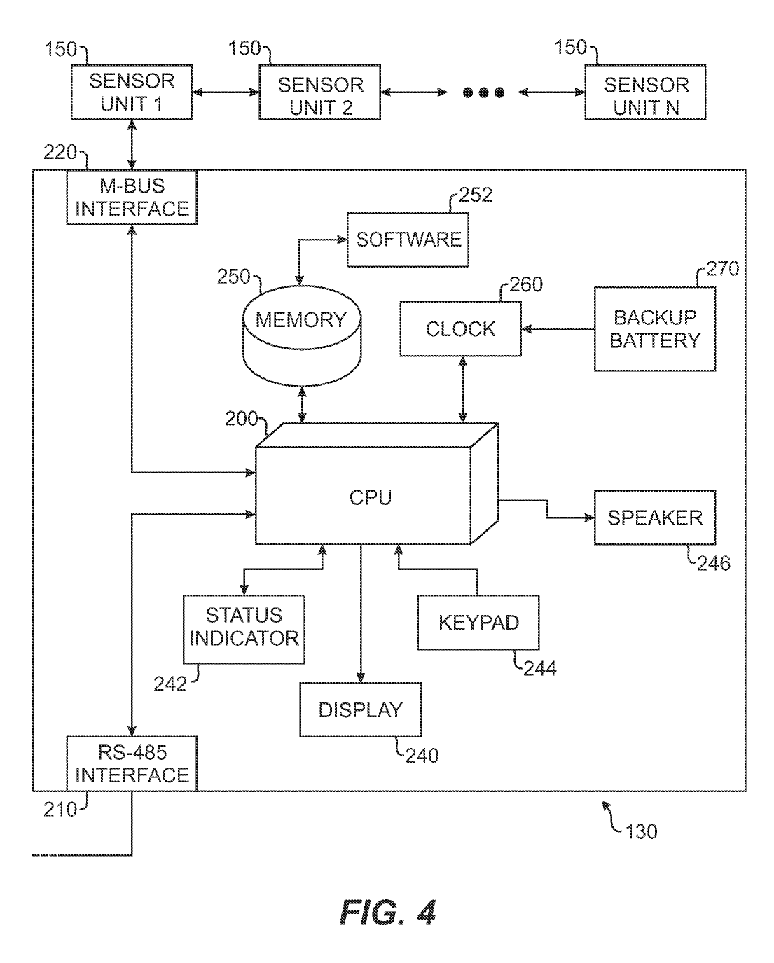

Referring to FIG. 4, the monitoring unit 130 of FIG. 3 is schematically illustrated in more detail. The monitoring unit 130 includes a central processing unit (CPU) 200, a Meter-BUS communication interface 220, a display 240, a status indicator 242, a key pad 244, a speaker 246, a memory 250, a clock 260, and a backup power supply 270. The prediction system 130 may or may not include the display 240, status indicator 242, keypad 244, and/or speaker 246 depending on the implementation.

In one embodiment, the CPU 200 includes a main microcontroller, such as the P89V51RD2 microcontroller from Phillips Semiconductors that has 64-kB Flash and 1024 bytes of data RAM. In addition, the CPU 200 includes a sensor microcontroller, such as the P87LPC767 microcontroller from Phillips Semiconductors

The memory 250 stores software 252 and other data for the monitoring unit 130. The software 252 includes instructions for managing the sensor units 150 connected to the monitoring unit 200 and can include an algorithm according to the teachings of the present disclosure for calculating a mold growth risk factor. The memory 250 is preferably an electrically erasable programmable read-only memory (EEPROM), such as the CAT24C161 from Catalyst Semiconductor that has a Precision Reset Controller and Watchdog Timer. The clock 260 is a Real-Time Clock (RTC), such as the PCF8563 from Phillips Semiconductors.

The display 240 is an LCD Display Panel HS162-4, which can display a plurality of characters. The key pad 244 preferably has a plurality of keys to perform various functions, such as making a selection, changing a selection, or navigating screens. Among a number of possible functions, for example, a user can use the keys 244 to enter information to the CPU 200 and to page through temperature and humidity readings, status displays of sensor positions, system fault displays, etc.

The power supply (not shown) for the unit 130 can be a battery or conventional power supply. For battery power, the unit 130 preferably uses circuits and components known in the art for maintaining low power consumption. The backup battery 270 can be a miniature Li-battery unit for system power off to keep the clock 260 working normally.

As noted previously, the communication interface 220 of the monitoring unit 130 is preferably based on the Meter-Bus (“M-Bus”) protocol to communicate with the sensor units 150. The sensor units 150 are connected in series and connected through one pair of lines to the M-Bus communication interface 220. The monitoring unit 130 can alternatively use the RS-485 communication protocol to communicate with the sensor units 150. In either case, the data format for communication from the CPU 200 to the sensor units 150 can include a sequence number, a command, a length (bytes) of the communication, data[0] . . . data[m], and a cyclic redundancy check (CRC) for error detection. Likewise, the data format for communication from sensor units 150 to the CPU 200 can include a sequence number, a status of the sensor module, a length (bytes) of the communication, data[0] . . . data[m], and a cyclic redundancy check (CRC) for error detection. One skilled in the art will appreciate that other embodiments of the monitoring unit 130 can use other protocols for the interface 220, including, but not limited to, a wireless interface and protocol. Moreover, depending on the implementation of the disclosed monitoring unit 130, the interfaces 220 may include a plurality of inputs/outputs for the various sensor units 150.

As alluded to previously, the monitoring unit 130 can be a stand-alone device or can be connected to a computer system or the like. To connect such a computer system, the monitoring unit 130 can include an RS-485 communication interface 210, The RS-485 communication interface 210 uses RS-485 communication protocol and can include a Maxim MAX1487 transceiver for RS-485 communication with a main control computer, such as discussed below with reference to the embodiment of FIG. 5.

Referring to FIG. 5, another embodiment of a mold prediction system 102 according to certain teachings of the present disclosure is schematically illustrated. The prediction system 102 electronically monitors an environment and determines a probability of mold growth in the environment. The prediction system 102 includes a master control unit 110 having a master control computer 112 and a communication hub 114. The communication hub 114 can be an RS-485 Hub connected to the master control computer 112 via an RS-232 connection 116. A plurality of monitoring units 130 and sensor units 150 are connected to the communication hub 114. In the present example, the monitoring units 130 and sensor units 150 are separated into a plurality of zones or areas 120 (e.g., zone . . . zone N), which can help organize the monitoring and reporting of mold growth in the environment. The environment can be a room, building, facility, or any location where monitoring of mold growth is desirable.

The monitoring units 130 are similar to the embodiments discussed above with reference to FIGS. 3 and 4. The monitoring units 130 are connected to the communication hub 114 via an RS-485 BUS 118. Each monitoring unit 130 has one or more sensor units 150 connected serially via an M-BUS.

The sensor units 150 are similar to the embodiments discussed above with reference to FIGS. 3 and 4. The sensor units 150 are distributed throughout the environment and can be located near a sink, food storage area, kitchen, windowsill, attic, closet, or anywhere that it is desirable to monitor for mold growth. Placement of the sensor units 150 depends on a number of factors, including, but not limited to, the type of environment being monitored, any equipment or other items located near the sensor units 150, the distance of the sensor units 150 from a potentially mold prone area, implementation specific criteria, any interference from other equipment, the potential for generating false readings, etc. One skilled in the art of monitoring temperature and humidity will appreciate these and other factors when distributing the sensor units 150 throughout the environment.

Using the standard of the RS-485 communication protocol and the hub 114, the master control computer 112 can be linked to numerous monitoring units 130, but the master control computer 112 preferably links to no more than two-hundred and fifty-five (255) monitoring units 130. In addition, each monitoring unit 130 can be linked to up to about one-hundred and twenty-eight (128) sensor units 150. Preferably, the maximum length of wiring from a given sensor unit 150 to the master control computer 112 does not exceed 1000-m.

During operation, the sensor units 150 collect data related to temperature and relative humidity in the environment. The monitoring units 130 gather the data from their associated sensor units 150. To track the collected data, the sensor units 150 and the monitoring units 130 have serial or identification numbers. The monitoring units 130 communicate collected data to the master control computer 112. In one embodiment, the monitoring units 130 only communicate collected temperature readings and humidity readings (and optionally time readings) to the master control computer 112, which calculates the mold grow risk factors. Alternatively, the monitoring units 130 calculate the mold growth risk factors and communicate collected temperature readings and humidity readings (and optionally time readings) along with the mold growth risk factors to the master control computer 112.

Software operating on monitoring unit 130 and/or the master control computer 112 is used to analyze the collected data and to generate warnings or perform other functions disclosed herein. For example, a user of the master control computer 112 and associated software can review the sensor readings and calculated mold growth risk factors for the various sensor units 150 and zones 120 of the environment. The software operating on the master control computer 112 can generate alarms when the risk factor of a given sensor unit 150 or zone 120 meets or exceeds a predetermined threshold. The software can also perform various known mathematical analyses on the readings of the sensor units 150. For example, the software can determine average readings and risk factors for a collection of sensor units 150 in a zone 120 and can forecast values for the risk factor using modeled values. The monitoring units 130 and the master control computer 112 may be capable of displaying similar information and performing similar functions.

Now that details related to how the monitoring units (130; FIG. 3-5) and sensor units (150; FIG. 3-5) collect readings of temperature, humidity, and time have been discussed, we now turn to a discussion how the collected data is analyzed. As discussed above, the monitoring unit (130; FIG. 3-5) and/or the master control computer (112; FIG. 5) can perform the functions of analyzing the collected data. During the analysis, an algorithm is used to determine a probability of mold growth for the environment using the temperature readings, the humidity readings, and the time readings. The algorithm is based on information associated with mold growth. Before discussing the algorithm in detail, we first discuss the forms of information associated with mold growth upon which the algorithm is based.

Referring to FIG. 6, graphs 300 and 350 illustrate isopleths for various species of mold. Graph 300 has a plurality of isopleths 320 that represent spore germination for various species of mold, and graph 350 has a plurality of isopleths 370 that represent mycelium growth for the various species of mold.

In graph 300, the spore germination isopleths 320 for the various species of mold are plotted against temperature (C) and relative humidity (%). As shown, the various species have spore germination isopleths 320 fall within different ranges of temperature and relative humidity. The graph 300 further includes an envelope 310, which is determined as a threshold for any spore germination to develop for the various species of mold. The area 330 of the graph 300 above or exceeding the values of the envelope 310 represents a Conducive State 330 conducive to spore germination for the various species of mold. Contrariwise, the area 340 of the graph 300 below or less than the values of the envelope 310 represents a Detrimental State 340 detrimental to spore germination for the various species of mold.

Similarly, in the graph 350, the mycelium growth isopleths 370 for the various species of mold are plotted against temperature (C) and relative humidity (%). As shown, the various species have mycelium growth isopleths 370 fall within different ranges of temperature and relative humidity. The graph 350 further includes an envelope 360, which is determined as a threshold for any mycelium growth to develop for the various species of mold. The area 380 of the graph 350 above or exceeding the values of the envelope 360 represents a Conducive State 330 conducive to mycelium growth for the various species of mold. Contrariwise, the area 390 of the graph 350 below or less than the values of the envelope 360 represents a Detrimental State 340 detrimental to mycelium growth for the various species of mold.

These graphs 300 and 350 plot the envelopes 310, 360 and isopleths 320, 370 based on a given time interval and substrate quality. Experimental data of relative humidity levels, temperatures, substrates, and time intervals for the various species of mold can be used to develop information for the disclosed system. The types of substrates may be particularly suited for a given environment in which the prediction system is intended to be installed. Alternatively, a general nutrient level of substrates may be used based on the circumstances. In addition, the information on isopleths and envelopes similar to those shown in FIG. 6 can be developed for various time intervals, such as a plurality of days. The information is then stored in the disclosed system and/or implemented into software for the disclosed system using various techniques known in the art.

By monitoring the temperature and relative humidity in a zone being monitored with sensors, the monitored conditions are analyzed using the software algorithm and stored information of the disclosed system. For example, if the monitored temperature is 25-degrees Celsius and the relative humidity is 75% for a given time interval and substrate quality (either general or specific), then the conditions may lie within Conducive States 330 380 of both graphs 300, 350 conducive to both spore germination and mycelium growth. By contrast, if the monitored temperature is 15-degrees Celsius and the relative humidity is 70%, then the conditions may lie within Detrimental States 340, 390 of both graphs 300, 350 detrimental to both spore germination and mycelium growth.

Based on which of the Conducive or Detrimental States the conditions fall and based on the length of time occurring within those conditions, the software algorithm of the disclosed system determines the probability of mold growth for the zone. In general, a longer period of time where conditions occur in Conducive States 330, 380 beyond the envelopes 310, 360 will correspond to greater potential for spore germination and mycelium growth. Likewise, the higher the conditions in Conducive States 330, 380 are beyond the envelopes 310, 360 will also correspond to greater potential for spore germination and mycelium growth. In contrast, a longer period of time where conditions occur in Detrimental States 340, 390 under the envelopes 310, 360 will correspond to less potential for spore germination and mycelium growth and potentially to elimination of existing mold. Likewise, the lower the conditions in Detrimental States 340, 390 are below the envelopes 310, 360 will also correspond to less potential for spore germination and mycelium growth and potentially to greater elimination of existing mold.

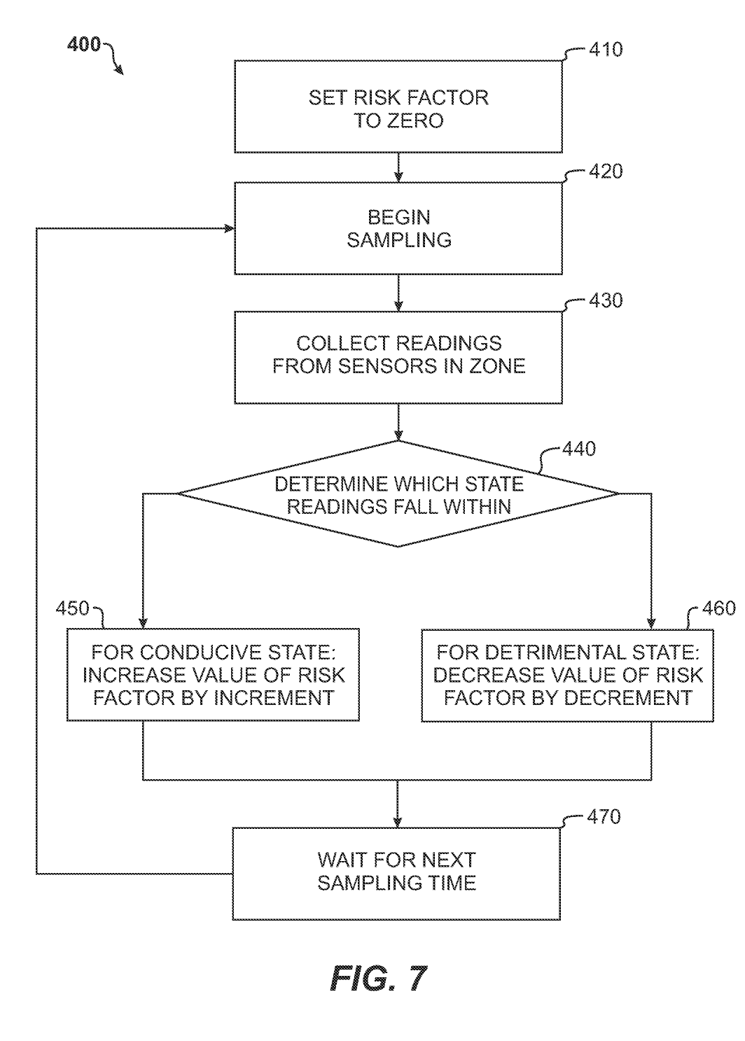

Accordingly, the software algorithm of the disclosed system is configured to use stored information similar to that shown in graphs 300, 350 to determine the probability of mold growth and potentially to control the mold growth in the environment. As will be appreciated, the stored information can be coded as part of the software algorithm as one or more formulas or can be implemented in searchable files stored in memory. Furthermore, a particular implementation may be tailored to monitor a common group of mold species or to monitor one or more specific mold species, and the software implementation can be tailored to monitor such species. Further details related to monitoring the conducive and detrimental states for spore germination and mycelium growth are discussed below with reference to FIG. 7.

Referring now to FIG. 7, an embodiment of an algorithm 400 for evaluating the conditions conducive and detrimental to mold growth is illustrated in flow chart form. As noted above, such an algorithm 400 can be incorporated into software for the disclosed system used to predict and provide early warning of potential mold growth. Among the four conditions (temperature, relative humidity, time, and materials/nutrients) influencing mold growth, the influence of the temperature, relative humidity, and time on mold growth are used in the present embodiment of the algorithm. For example, the algorithm has an equation that incorporates the dependence of at least the temperature and relative humidity on time. However, each of the four conditions that determine mold growth can be considered in the algorithm 400. For example, the quality of various substrates can be used in the algorithm because the sensors are placed in various places in the environment having known materials, such as carpet, wallpaper, wood structures, tile, cloth, PVC pipe, etc. Therefore, the particular attributes of the substrate in the area of the sensor (e.g., the substrates level of nutrients conducive to mold growth) can be used to further tailor the determination of mold growth near the sensor.

In the algorithm 400, an initial value of the probability or risk factor for mold growth in a zone is set to zero (Block 410). In general, the risk factor for mold growth can be allowed to range from 0 to 1. If the value of the risk factor is negative after performing the computations discussed below, the risk factor can be set to zero. Similarly, if the value of the risk factor is greater than 1 after performing the computations discussed below, the risk factor can be set to 1. Adjusting the risk factor in this manner will allow for reporting the value of the risk factor in the form of a percentage from 0 to 100%.

The system begins sampling the sensors for temperature and humidity readings (Block 420). The frequency of the sampling can be suited for the particular implementation. For example, the sampling can occur at predetermined time intervals, such as every 10-minutes, so that the risk factor for the zone can be regularly monitored and updated. The system receives or obtains the readings of the temperature and relative humidity from the environment (Block 430). For example, the sensors in a zone detect the temperature and relative humidity levels at discrete times, and the readings are communicated to the central processing unit via the communication interface. It will be appreciated that a plurality of zones can be simultaneously monitored, monitored in staggering intervals, etc. In addition, it will be appreciated that the frequency of monitoring can be varied.

The system determines whether the monitored readings fall within a state conducive to mold growth or within a state detrimental to mold growth. The “mold growth” can refer to only spore germination, only mycelium growth, or both spore germination and mycelium growth, such as described above with reference to FIG. 6. It will be appreciated that various known mathematical techniques can be used to process data to determine the risk factor for mold growth. For example, known mathematical techniques, such as correlation, interpolation, curve fitting, history data matching, and neural networks, can be used.

If the condition of the readings fall within a Conducive State for mold growth, the conditions will contribute a positive value to the risk factor for mold growth based on the time it takes to grow mold (e.g., for spore germination and/or mycelium growth to occur). Therefore, the sampling frequency or the predetermined interval between readings is used to determine passage of time. Then, the risk factor is increased by an increment based upon the value of the conditions, duration in the current conditions, and the predetermined amount of time and levels conducive to the plurality of species or one or more specific species of mold being monitored (Block 450).

In one embodiment of an equation for incrementing the risk factor, the risk factor at current sampling time equals the risk factor at the previous sampling time plus an increment occurring in the duration from the past sampling period. The increment is a positive value inversely proportion to the time it takes to grow mold from predetermined experimental data. For example, if the Conducive State indicates that it takes X days to grow one or more species of mold under certain conditions, and the sampling rate is Y hours, then the increment is based on the equation:
Increment=Y/(24×)

If the conditions in the readings fall within a Detrimental State, the conditions will contribute a negative value to the risk factor for mold growth based on the time it takes to stop, reverse, or eliminate mold growth (e.g., stop spore germination and/or stop or kill mycelium growth). Then, the risk factor is decreased by an decrement based upon the value of the conditions, duration in the current conditions, and the predetermined amount of time and levels detrimental to the plurality of species or one or more specific species of mold being monitored (Block 460).

In one embodiment of an equation for decreasing the risk factor, the risk factor at the current sampling time equals the risk factor at the previous sampling time plus any decrement occurring in the duration from the past sampling period. The decrement is a negative value and can be based on the exponential function:
Decrement=−Ae^{-(BT+CH)}

T represents the temperature reading, and H represents relative humidity reading. The parameters A, B, and C are non-negative values determined from the predetermined experimental data for the group of mold species or one or more specific mold species being monitored.

After adjusting the risk factor to reflect recent conditions monitored in the environment, the system waits for the next sampling time (Block 470). When the next sampling time arrives, the system returns to Block 420 to begin a new sampling cycle to update the risk factor or probability of mold growth.

As discussed previously with reference to FIGS. 3-5, the sensor units 150 generate temperature and relative humidity readings at a plurality of intervals, and the monitoring units 130 collect these readings. The collected readings are then communicated to the master control unit 110, which analyzes the readings. To analyze the readings, the master control unit 110 can track historical data and maintain running calculations of the risk factor for mold growth in various zones 120 and various locations of particular sensor units 150 of the system 100 in the environment. This historical data can be displayed on the master control computer 112 using software in various forms, such as using graphs.

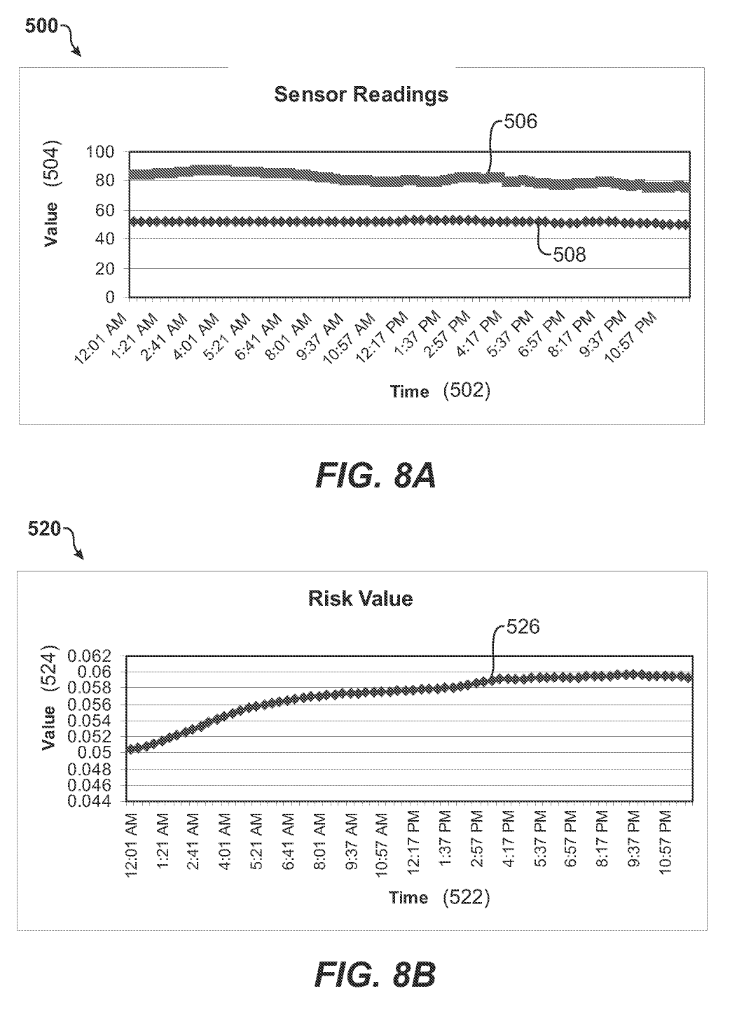

Referring to FIGS. 8A-8B, examples of sensor readings and calculated risk values are graphically illustrated. Graph 500 of FIG. 8A shows relativity humidity readings 506 and temperature readings 508 for a day of readings from one sensor unit. Humidity readings 506 are graphed as a function of time 502 and values 504 in units of percentage of relative humidity. The values 504 for the humidity readings 506 range from about 77 to 87-% relative humidity. Temperature readings 508 are graphed as a function of time 502 and values 504 in Celsius. The values 504 for the temperature readings 508 range from about 50 to 52-degrees Celsius. Graph 520 of FIG. 8B shows the calculated value of the mold growth risk factor 526 based on the sensor readings of FIG. 8A. The risk factor 526 is graphed as a function of time 522 and values 524. As shown, the risk factor 526 generally increases as time passes and as the temperature readings (508) and relative humidity readings (506) moderately increase and decreases during the day.

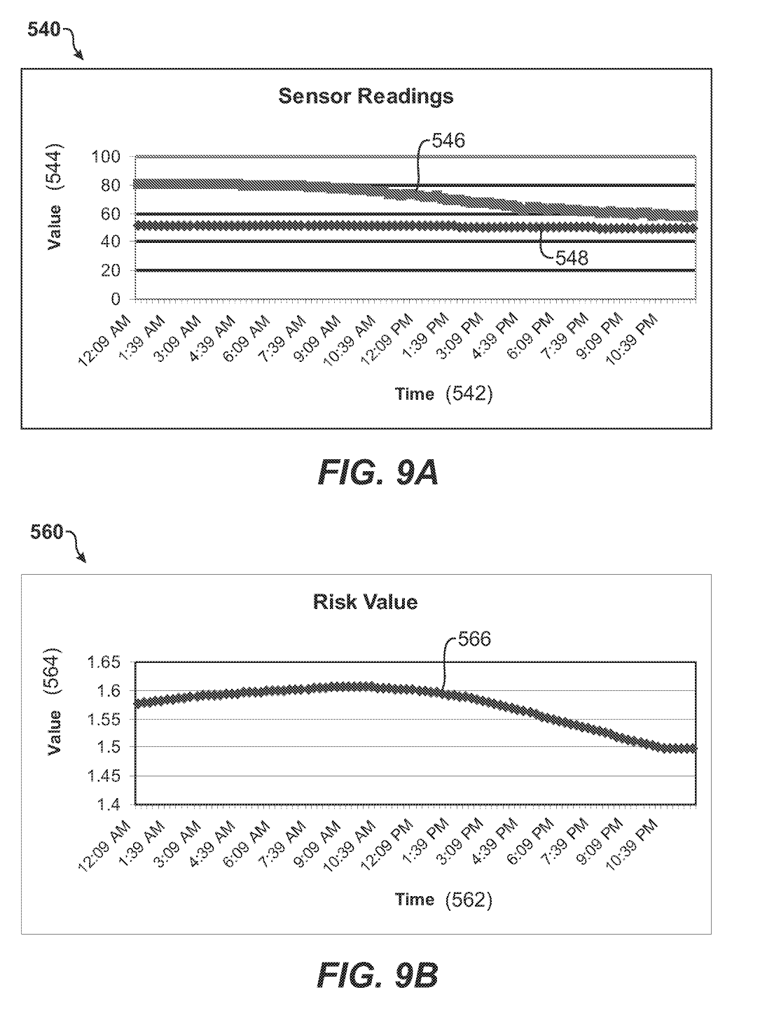

Referring to FIGS. 9A-9B, another example of sensor readings and calculated risk values are graphically illustrated. Graph 540 of FIG. 9A shows relative humidity readings 546 and temperature readings 548 for a day of readings. Humidity readings 546 are graphed as a function of time 542 and values 544 in units of percentage of relative humidity. The values 544 for the humidity readings 546 range from about 57 to 81-% relative humidity. Temperature readings 548 are graphed as a function of time 542 and values 544 in Celsius. The values 544 for the temperature readings 548 range from about 49 to 51-degrees Celsius. Graph 560 of FIG. 9B shows the calculated value of the mold growth risk factor 566 based on the sensor readings of FIG. 9A. The risk factor 566 is graphed as a function of time 562 and value 564. As shown, the risk factor 566 generally decreases as time passes, as the temperature readings (548) remain relatively constant, and as the relative humidity readings (546) decrease during the day.

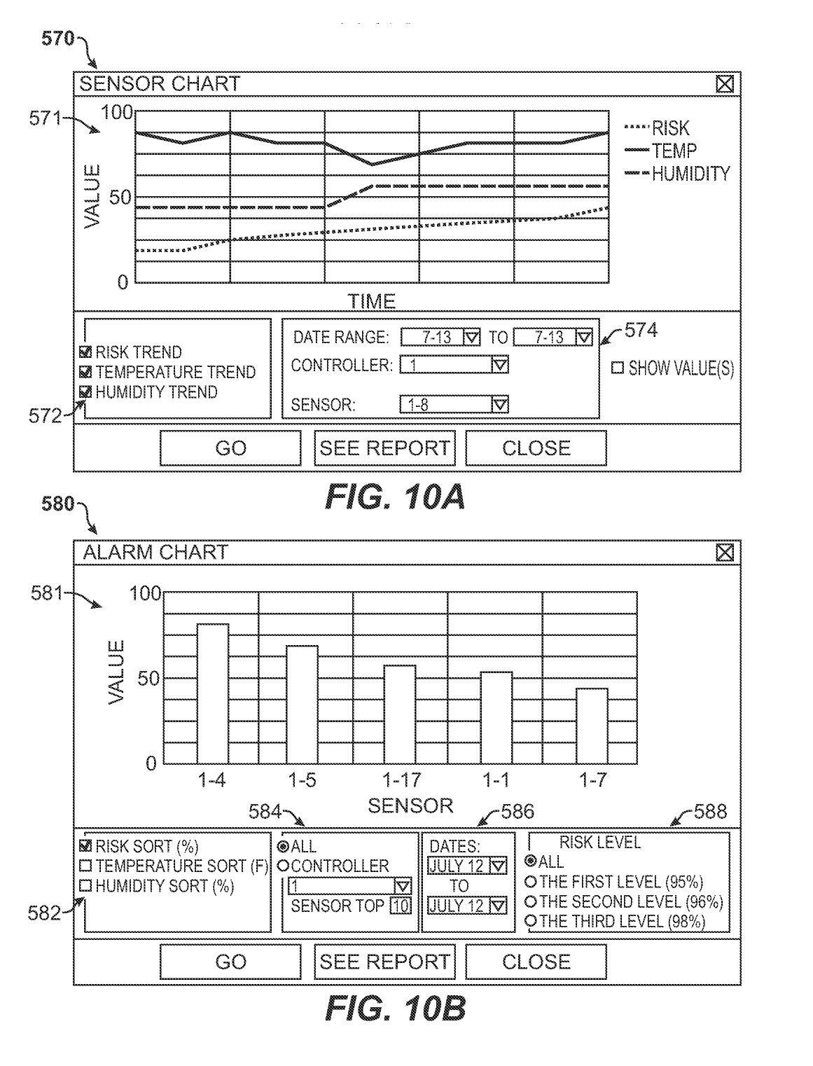

Referring to FIGS. 10A-10B, example screens 570 and 580 for a graphical user interface of a master control computer (112; FIG. 5) are illustrated. Screen 570 of FIG. 10A shows a graph 571 of selected trends 572 of a selected sensor 574. For the trends 572, the user can select to display risk, temperature, and/or humidity. To select the sensor 574, the user can specify the controller number (i.e., the ID for a monitoring unit) and the sensor number (i.e., the ID number of a sensor unit). The user can also specify a date range.

Screen 580 of FIG. 10B shows a graph 581 of highest values for selected sensors. In fields 582, the user can sort the display on the graph 581 by risk, temperature, and/or humidity. In fields 584, the user can select to generate the graph from all of the sensors or only some of those associated with a designated controller (i.e., monitoring unit). In fields 586, the user can select date ranges. Finally, the user can specify what risk levels to display including all or some percentage in fields 588. One skilled in the art will appreciate that a user interface of a master control computer can have these and other screens.

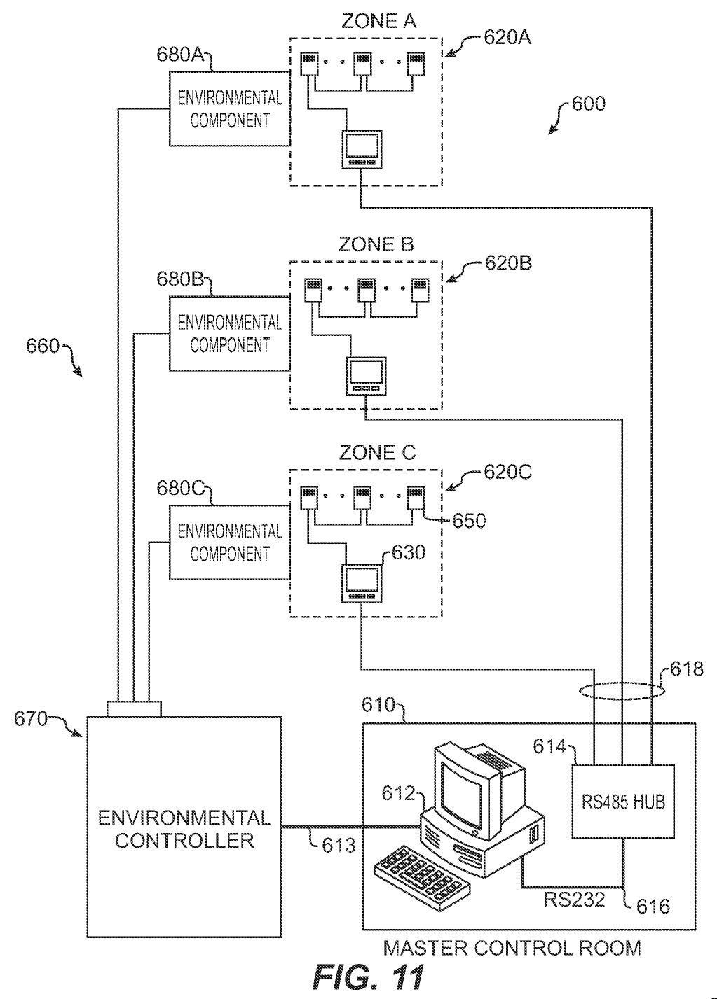

In addition to monitoring, displaying, and analyzing the readings and risk factor information, the mold growth prediction system of the present disclosure can proactively alter aspects of the environment to control or reduce the potential for mold growth in the environment. Referring to FIG. 11, a prediction system 600 and an environmental system 660 according to one embodiment of the present disclosure are illustrated. The prediction system 600 is integrated with the environmental system 660. The prediction system 600 can be substantially similar to other embodiments disclosed herein. For example, the prediction system 600 includes a master control unit 610 having a master control computer 612 connected to a RS-485 Hub 614 via a RS-232 connection 616. The hub 614 connects to various zones 620A-C distributed in the environment via RS-485 connections 618. The zones 620A-C include monitoring units 630 and sensor units 650 similar to those discussed previously. The master control unit 610 receives temperature and humidity readings of the various zones 620A-C and determines the risk factor or probability of mold growth for the sensor units 650 and the various zones 620A-C.

Rather than merely indicate the risk factors (e.g., display the risk factors for a user or produce an alarm), the master control unit 610 further includes an interface 613 with an environment controller 670 of the environmental system 660 for the environment. Although the prediction system 600 and environmental controller 670 are shown as separate entities or units in the present embodiment, it will be appreciated that the monitoring and environmental control of the present disclosure can be implemented within a single entity or unit or within more than two entities or units.

The environmental controller 670 is coupled to a plurality of environmental components or units 680A-C, which can be heating, ventilation, and air-conditioning (HVAC) components, dehumidifiers, humidifiers, fans, and other components coupled to the environmental controller 670 that can alter the environmental conditions of the zones 620A-C. The environmental controller 670 controls the various components 680A-C. Although each zone 620A-C of the environment is shown with its own environmental component 680 in the present embodiment, it will be appreciated that various zones of an embodiment can have more than one environmental component 680 or one environmental component 680 can service more than one zone depending on the particular implementation. The environmental controller 670 can control the heating, ventilation, and air conditioning of the environment by operating the various environmental components 680, such as operating air-conditioning to lower the temperature, operating air-conditioning to reduce relative humidity, operating heating to raise the temperature, operating a dehumidifier to reduce the relative humidity, diverting airflow, distributing airflow, etc.

During operation, the prediction system 600 receives temperature and humidity readings from the sensor units 650 in the zones 620A-C of the environment. Based on the readings, the prediction system 600 determines the risk factor or probability of mold growth in the zones 620A-C over time using techniques disclosed herein. When a zone (e.g., zone 620A) develops an unacceptable risk factor or probability of mold growth, the prediction system 600 determines what combination of conditions (e.g., temperature, humidity, time) would be detrimental to any mold growth in the zone 620A and could potentially stop, reverse, or kill any current mold growth in the zone 620A. The prediction system 600 relays the combination of detrimental conditions (temperature, humidity, time) to the environmental controller 670. In turn, environmental controller 670 controls the environmental component 680A associated with zone 620A with operational parameters consistent with the combination of detrimental conditions (e.g., temperature, humidity, time) for addressing the mold growth in zone 620A.

For example, the risk or probability of mold growth in zone 620A may reach 75%, the current temperature reading may be T_{Current}, and the current relative humidity reading may be H_{Current}. Based on the species of mold being monitored in zone 620A, the current readings (T_{Current}, H_{Current}), and the techniques for addressing mold growth disclosed herein, the prediction system 600 may determine that a new temperature level of T_{New} and new humidity level of H_{New} applied to the zone 620A for a period of time could potentially address the mold growth in zone 620A. At least the new temperature level and time interval can be sent to the environmental controller 670, which can then operate the HVAC component 680A associated with zone 620A to maintain the desired temperature for the time interval. The environmental controller 670 may have its own sensors for monitoring the time and temperature of the zone.

Alternatively, the prediction system 600 and environmental system 660 can operate in a cooperative relationship. For example, the prediction system 600 can send only a new temperature level for zone 620A to the environmental controller 670, which can then operate the HVAC component 680A associated with zone 620A to maintain the desired temperature. The environmental controller 670 may have its own sensors for monitoring the time and temperature of the zone, or it can use the sensors 650 of the prediction system 600. The prediction system 600 then continues monitoring the zone 620A with the sensor units 650 to determine when and if the desired new temperature is met. The current operation can be maintained until the time interval expires and the prediction system 600 instructs the environmental controller 670 to cease its proactive operation. Alternatively, the current operation can be maintained until the prediction system 600 detects the desired relative humidity or determines a particular reduction in the risk factor and instructs the environmental controller 670 to cease its proactive operation of the HVAC component 680A.

In one possible extension of the integrated prediction system 600 and environmental system 660, the temperature sensors within the sensor units 650 can be used to detect significantly elevated temperatures caused by a potential fire in the environment. The master control unit 610 can be configured to detect such significantly elevated temperature readings and can communicate an alarm to a security system or fire alarm system of the environment.

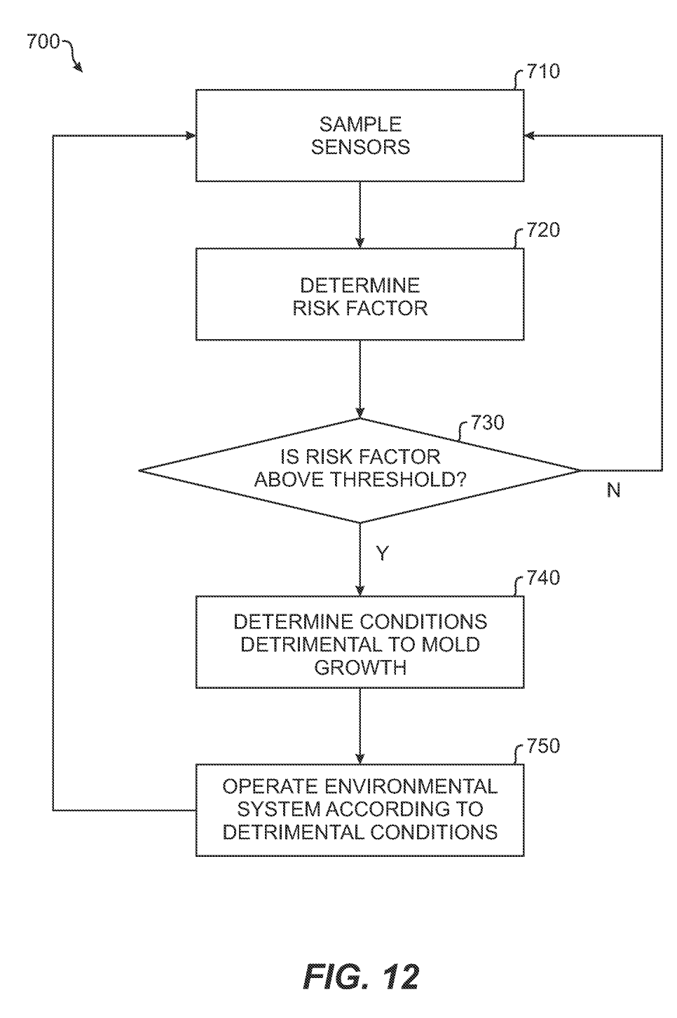

Referring to FIG. 12, an embodiment of an algorithm 700 for interfacing a prediction system with an environmental system to control mold growth is illustrated in flow chart form. As discussed above in the embodiment of FIG. 11, the disclosed prediction system can be integrated with or coupled to the environmental system. Based on the determinations made by the prediction system with respect to mold growth, the prediction system operates in conjunction with the environmental system to address or control the growth of mold in the environment.

To begin, the prediction system samples the sensors (Block 710) and determines the risk factor or probability for mold growth (Block 720) in a manner similar to that described above with reference to FIG. 7. A determination is then made whether the risk factor is above threshold criteria (Block 730). For example, the threshold criteria can be a particular value of the risk factor (e.g., 75%) or the threshold criteria can be a particular value of the risk factor (e.g., 75%) for a particular amount of time (e.g., 24 hours). Other than the use of a threshold for the determination, it will be appreciated that various other forms of criteria can be employed. For example, issues related to hysterisis may be integrated into the determination of Block 730. In addition, the threshold criteria may have more than one level of severity. For example, a first level for the threshold criteria may recognize a low level of risk for mold growth, a second level for the threshold criteria may recognize a medium level of risk for mold growth, and third level for the threshold criteria may recognize a high level of risk for mold growth. Each of these levels can have corresponding levels of action for the disclosed system to implement as discussed below.

If the risk factor does not meet or exceed the threshold criteria at Block 730, then the system returns to sampling the sensors according to Block 710. If, however, the risk factor does meet or exceed the threshold criteria at Block 730, then the system determines which detrimental conditions (temperature, relative humidity, and/or time) would be detrimental to mold growth for the environment under the circumstances. For example, operation of the air conditioning unit for a certain amount of time in the zone may reduce the temperature and relative humidity to a level that will stop, reverse, or kill any existing mold growth within the zone. Finally, the environmental system is operated according to the detrimental conditions to address or control the mold growth in the zone (Block 750). The system can then return to sampling the sensors in Block 710 so that the system operates in a looped operation.

The foregoing description of preferred and other embodiments is not intended to limit or restrict the scope or applicability of the inventive concepts conceived of by the Applicants. In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

What is claimed is:

1. A mold growth prediction system for an environment, comprising:
a processing unit configured to:
obtain temperature data, humidity data, and time data for the environment, and
determine a probability of mold growth for the environment based on the temperature data, the humidity data, and the time data.

2. The system of claim 1, wherein a computer includes the processing unit.

3. The system of claim 1, wherein a monitoring unit includes the processing unit.

4. The system of claim 3, wherein the monitoring unit comprises a display and one or more user controls coupled to the processing unit.

5. The system of claim 1, further comprising at least one first sensor generating the temperature data of the environment and communicating the temperature data to the processing unit.

6. The system of claim 5, further comprising at least one second sensor for generating the humidity reading of the environment and communicating the humidity data to the processing unit.

7. The system of claim 6, wherein the at least one first sensor and the at least one second sensor are housed in an integrated sensor unit.

8. The system of claim 7, wherein the processing unit is communicatively coupled to the integrated sensor unit via a first interface and is communicatively coupled to a computer via a second interface, the processing unit collecting the temperature and humidity data from the sensors of the sensor unit via the first interface and communicating the collected temperature and humidity data to the computer via the second interface.

9. The system of claim 8, wherein the first interface comprises a Meter-Bus interface, and wherein the second interface comprises an RS-485 interface.

10. The system of claim 9, further comprising a hub connected to the RS-485 interface of the processing unit and connected to the computer via an RS-232 connection.

11. The system of claim 1, wherein the processing unit comprises:
an interface for obtaining the temperature data and the humidity data,
a memory for storing an algorithm to determine the probability of mold growth, and
a processor communicatively coupled to the interface and the memory, the processor processing the temperature data, the humidity data, and the time data according to the algorithm to determine the probability of mold growth based on the temperature data, the humidity data, and the time data.

12. The system of claim 11, wherein the interface comprises a wired interface using a communication protocol selected from the group consisting of RS-485, RS-232, and Meter-BUS.

13. The system of claim 1, wherein the processing unit comprises an algorithm for determining the probability of mold growth based on the temperature data, the humidity data, and the time data.

14. The system of claim 13, wherein the algorithm is configured to:
determine whether the temperature data and the humidity data fall within conditions detrimental to mold growth,
determine a decrement value based on the detrimental conditions, and
decrease a previous probability of mold growth by the decrement value to produce a current probability of mold growth.

15. The system of claim 13, wherein the algorithm is configured to:
determine whether the temperature data and the humidity data fall within conditions conducive to mold growth,
determine an incremental value based on the conducive conditions, and increase a previous probability of mold growth by the incremental value to produce a current probability of mold growth.

16. The system of claim 1, further comprising an environmental system communicatively coupled to the processing unit, the environmental system configured to control at least one condition of the environment based on the probability of mold growth to reduce the probability of mold growth for the environment.

17. An electronic mold growth prediction method for an environment, comprising:
storing information on mold growth, the information at least defined by temperature, humidity, and time;
obtaining temperature data, humidity data, and time data for the environment; and
determining a probability of mold growth with the stored information based on the temperature data, the humidity data, and the time data.

18. The method of claim 17, wherein the act of obtaining temperature data, humidity data, and time data for the environment comprises:
receiving a temperature reading from a temperature sensor; and
receiving a humidity reading from a humidity sensor.

19. The method of claim 17, wherein the act of storing the information on mold growth comprises storing an equation defining an envelope based on temperature, humidity, and one or more species of mold, the envelope substantially separating conditions detrimental to mold growth from conditions conducive to mold growth for the species of mold.

20. The method of claim 17, wherein the act of determining the probability of mold growth with the stored information comprises:
determining whether the temperature data and the humidity data fall within conditions detrimental to mold growth;
determining a decrement value based on the detrimental conditions; and
decreasing a previous probability of mold growth by the decrement value to produce a current probability of mold growth.

21. The method of claim 17, wherein the act of determining the probability of mold growth with on the stored information comprises:
determining whether the temperature data and the humidity data fall within conducive conditions to mold growth;
determining an incremental value based on the conducive conditions; and
increasing a previous probability of mold growth by the incremental value to produce a current probability of mold growth.

22. The method of claim 17, further comprising controlling at least one condition of the environment based on the probability of mold growth to reduce the probability of mold growth for the environment.

23. The method of claim 22, wherein the act of controlling the at least one condition of the environment comprises controlling at least a humidity level of the environment.

24. A mold growth prediction system for an environment, comprising:
means for obtaining temperature data, humidity data, and time data for the environment; and
means for determining a probability of mold growth for the environment based on the temperature data, the humidity data, and the time data.

25. The system of claim 24, further comprising means for generating the temperature data for the environment.

26. The system of claim 24, further comprising means for generating the humidity data for the environment.

27. The system of claim 24, further comprising means for generating the time data for the environment.

28. The system of claim 24, wherein the means for obtaining the temperature data for the environment comprises means for interfacing with at least one sensor in the environment.

29. The system of claim 24, wherein the means for determining a probability of mold growth for the environment based on the temperature data, the humidity data, and the time data comprises:
means for determining whether the temperature data and the humidity data fall within the detrimental conditions to mold growth;
means for determining a decrement value based on the detrimental conditions; and
means for decreasing a previous probability of mold growth by the decrement value to produce a current probability of mold growth.

30. The method of claim 24, wherein the means for determining a probability of mold growth for the environment based on the temperature data, the humidity data, and the time data comprises:
means for determining whether the temperature data and the humidity data fall within conducive conditions to mold growth;
means for determining an incremental value based on the conducive conditions; and
means for increasing a previous probability of mold growth by the incremental value to produce a current probability of mold growth.

31. The method of claim 24, further comprising means for controlling at least one condition of the environment based on the probability of mold growth to reduce the probability of mold growth for the environment.
